(19) United States
(12) Patent Application Publication
(10) Pub. No.: US 2023/0222312 A1
(43) Pub. Date: Jul. 13, 2023

(54) METHOD FOR EVALUATING OF BIOAVAILABILITY OF ORGANIC NITROGEN IN SEWAGE

(71) Applicant: Nanjing University, Nanjing (CN)

(72) Inventors: Haidong HU, Nanjing (CN); Huazai CHENG, Nanjing (CN); Kewei LIAO, Nanjing (CN); Jinfeng WANG, Nanjing (CN); Bing WU, Nanjing (CN); Hongqiang REN, Nanjing (CN)

(21) Appl. No.: 17/588,221

(22) Filed: Jan. 29, 2022

(30) Foreign Application Priority Data
Dec. 28, 2021 (CN) 202111627228.X

(51) Int. Cl. G06N 3/00; G16C 20/70; G16C 20/30; G01N 33/18

(52) U.S. Cl. CPC G06N 3/002; G01N 33/18; G16C 20/30; G16C 20/70

(57) ABSTRACT

A method for evaluating bioavailability of organic nitrogen in sewage through machine learning, includes: collecting the molecular composition information and bioavailability data of organic nitrogen in a sewage sample; establishing a model for predicting bioavailability of organic nitrogen in sewage through machine learning; measuring the molecular composition information of organic nitrogen in sewage from a target sewage plant; and predicting, according to the model, the bioavailability of the organic nitrogen in the sewage from the target sewage plant.

### CROSS-REFERENCE TO RELATED APPLICATIONS

Pursuant to 35 U.S.C.§ 119 and the Paris Convention Treaty, this application claims foreign priority to Chinese Patent Application No. 202111627228.X filed Dec. 28, 2021, the contents of which, including any intervening amendments thereto, are incorporated herein by reference. Inquiries from the public to applicants or assignees concerning this document or the related applications should be directed to: Matthias Scholl PC., Attn.: Dr. Matthias Scholl Esq., 245 First Street, 18th Floor, Cambridge, MA 02142.

### BACKGROUND

The disclosure belongs to the field of sewage treatment, and more particularly to a method for evaluating bioavailability of organic nitrogen in sewage through machine learning.

Conventionally, the bioavailability of organic nitrogen in sewage is measured by algae bioassay. An algae inoculation solution, a sludge mixed solution, and a sewage sample are mixed and then cultured for 14 to 28 days in an artificial climate chamber, and the bioavailability of organic nitrogen is represented by the percentage of the organic nitrogen consumed during the culture process in the total organic nitrogen. However, this evaluation method has some disadvantages such as long culture time and strict culture condition, and is thus difficultly applied in continuous monitoring of the bioavailability of organic nitrogen in sewage from sewage treatment plants.

### SUMMARY

The disclosure provides a method for evaluating bioavailability of organic nitrogen in sewage through machine learning, the method comprising:
(1) collecting the molecular composition information and bioavailability data of organic nitrogen in a sewage sample;
(2) establishing a model for predicting bioavailability of organic nitrogen in sewage through machine learning;
(3) measuring the molecular composition information of organic nitrogen in sewage from a target sewage plant; and
(4) predicting, according to the model established in (2), the bioavailability of the organic nitrogen in the sewage from the target sewage plant.

In a class of this embodiment, the molecular composition information of organic nitrogen in the sewage sample comes from data measured by a Fourier transform ion cyclotron resonance mass spectrometer, and the bioavailability of organic nitrogen in sewage comes from data measured by algae biological culture.

In a class of this embodiment, the model for predicting bioavailability of organic nitrogen in sewage is established by a random forest model in machine learning, which comprises:
(a) calculating an organic nitrogen molecular parameter, and performing data standardization by using the organic nitrogen molecular parameter as a feature value;
(b) searching the best number of features, and determining features to be deleted by feature ranking;
(c) dividing a data set to obtain a training set, a validation set, and a test set, training the model by using the training set, and optimizing model parameters by using the validation set; and
(d) selecting a best optimal model parameter to train the model to obtain a prediction model, and evaluating the performance of the prediction model by using the test set.

In a class of this embodiment, in (a), the organic nitrogen molecular parameter as the feature value comprises: molecular parameters of all organic nitrogen molecules, and organic nitrogen molecular parameters of seven molecule categories; and, the molecular parameters of all organic nitrogen molecules comprise: a mass-to-charge ratio m/z, a number C of carbon atoms, a number H of hydrogen atoms, a number O of oxygen atoms, a number N of nitrogen atoms, a ratio O/C of the number of oxygen atoms to the number of carbon atoms, a ratio H/C of the number of hydrogen atoms to the number of carbon atoms, a number DBE of double bond equivalents, a ratio DBE/H of the number of double bond equivalents to the number of hydrogen atoms, a ratio DBE/O of the number of double bond equivalents to the number of oxygen atoms, a ratio (DBE-O)/C of a difference between the number of double bond equivalents and the number of oxygen atoms to the number of carbon atoms, an average value of a nominal oxidation state of carbon (NOSC) of all organic nitrogen molecules, and strength weighted average values of molecular parameters, which are equal to a sum of products of respectively multiplying corresponding relative peak strength of molecules by m/z, C, H, O, N, O/C, H/C, DBE, DBE/H, DBE/O, (DBE-O)/C and NOSC.

In a class of this embodiment, the seven molecule categories are: lipids, proteins/amino sugars, carbohydrates, unsaturated hydrocarbons, lignin, tannins and condensed aromatics; the screening conditions for lipids are as follows: O/C < 0.2 and 1.7 < H/C < 2.2; the screening conditions for proteins/amino sugars are as follows: 0.2 < O/C < 0.6, 1.5 < H/C < 2.2 and N/C ≥ 0.05; the screening conditions for carbohydrates are as follows: 0.6 < O/C < 1.0 and 1.5 < H/C < 2.2; the screening conditions for unsaturated hydrocarbons are as follows: O/C<0.1, 0.7<H/C<1.5; the screening conditions for lignin are as follows: 0.1 < O/C < 0.6, 0.6 < H/C < 1.7, and the modified aromaticity index AImod < 0.67; the screening conditions for tannins are as follows: 0.6 < O/C < 1.0, 0.5 < H/C < 1.5 and the modified aromaticity index AImod < 0.67; and, the screening conditions for condensed aromatics are as follows: O/C < 1.0, 0.3 < H/C < 0.7 and the modified aromaticity index AImod ≥ 0.67.

In a class of this embodiment, the organic nitrogen molecular parameters of seven molecule categories comprise: the mass-to-charge ratio m/zi, the number DBEi of double bond equivalents and the average value of the nominal oxidation state of carbon NOSCi of organic nitrogen molecules of the seven molecule categories, the proportion Numi of the number of molecules in each category, and strength weighted average values of molecular parameters, which are equal to a sum of products of respectively multiplying corresponding relative peak strength of molecules by m/zi, DBEi and NOSCi, which i represents the molecule categories.

In a class of this embodiment, data standardization is performed on the feature value by the following calculation formula:

$$z = \frac{\left(x - u\right)}{s} ;$$

where z is a standardized feature value, x is an original feature value, u is an average value of the feature value, and s is a standard deviation of the feature value.

In a class of this embodiment, to search the best number of features, a recursive feature elimination algorithm with cross-validation is used, NGBoost is selected as a learning estimator, and a determination coefficient R² is used as a cross-validation scoring basis; and, every time one feature number is removed from the current feature set, the feature elimination process is recursively circulated in the updated feature set until the cross-validation score of the model decreases due to the feature elimination, and features to be deleted are determined by feature ranking.

In a class of this embodiment, the data set is randomly divided into a training set and a test set at a ratio of 9:1, m samples are randomly selected from the given training set to construct a sample set, k attributes are randomly selected from an attribute set of each node of a base decision tree by using a decision tree as a base learner, and the best attribute is selected from the k attributes for division; sampling is performed for T times to construct a sample set containing m training samples, and one decision tree is trained based on each sample set; and, a random forest model is formed based on T decision trees, and the final predicted value of the random forest model can be expressed as:

$${f}^{⌣} \left(x\right) = \frac{1}{T} {\sum }_{i = 1}^{T} T \left(x\right) ;$$

where f̌(x) is the final predicted value of the random forest model, T is the number of decision trees, and T(x) is the output value of each decision tree. The training set employs a 5-fold cross-validation mode to adjust model parameters and train the model, and the adjusted model hyper-parameters are evaluated on the validation set.

In a class of this embodiment, the random forest parameters to be adjusted and the ranges thereof are as follows: the number of base decision trees is 100 to 10000, the maximum depth of the decision tree is 5 to 55, the minimum impurity reduction threshold is 0.0 to 0.1; the parameters are randomly matched and combined to output the best parameter combination; and, based on the randomly matched best parameter combination, a number of values are selected in the parameter proximity range, and all possible combinations of parameters are traversed to output the best parameter combination.

In a class of this embodiment, the best model parameter is selected to train the model to obtain a prediction model, and the performance of the prediction model is evaluated by using the test set; and, the determination coefficient R² and the root mean square error RMSE are selected as evaluation indexes, where the calculation formula of R² is:

$${R}^{2} \left(y , {y}^{⌣}\right) = 1 - \frac{{\sum }_{i = 1}^{n} {\left({y}_{i} - {{y}^{⌣}}_{i}\right)}^{2}}{{\sum }_{i = 1}^{n} {\left({y}_{i} - \overline{y}\right)}^{2}} ;$$

the calculation formula of the RMSE is:

$$R M S E = \sqrt{\frac{1}{n} {\sum }_{i = 1}^{n} {\left({{y}^{⌣}}_{i} - {y}_{i}\right)}^{2}} ;$$

where yᵢ is a real value, y̌ᵢ is a predicted value,

$$\overline{y} = \frac{1}{n} {\text{¡}}_{i = 1}^{n} {y}_{i} ,$$

, and n is a total number of sewage samples.

In a class of this embodiment, using the model to predict the bioavailability of organic nitrogen in sewage comprises:
(a) measuring the molecular composition information of organic nitrogen in sewage by a Fourier transform ion cyclotron resonance mass spectrometer;
(b) calculating a desired feature value, and performing data standardization on the feature value; and
(c) inputting the data in (b) into the prediction model, and running the prediction model to obtain an output vale, so that the prediction of the bioavailability of organic nitrogen in sewage can be completed.

The following advantages are associated with the method for evaluating bioavailability of organic nitrogen in sewage through machine learning of the disclosure.

(1) In the disclosure, few test water samples are required for evaluating the bioavailability of organic nitrogen in sewage, and the algae biological culture is not required, so that the test period can be greatly shortened. In addition, the bioavailability of organic nitrogen in sewage can be predicted immediately after the molecular composition information of organic nitrogen is obtained, and the average accuracy of prediction can reach above 90%.

(2) In the disclosure, the method for evaluating the bioavailability of organic nitrogen in sewage is easy to operate, and the bioavailability of organic nitrogen in sewage can be obtained only by inputting the molecular composition information of organic nitrogen in the sewage sample into the trained machine learning model, so that the tedious experimental operation processes such as algae biological culture are avoided.

### BRIEF DESCRIPTION OF THE DRAWINGS

FIG. 1 is a graph of the determined best number of features in machine learning according to the disclosure;

FIG. 2 is a schematic diagram of division of the data set of the established prediction model according to the disclosure;

FIG. 3 is a schematic structure diagram of the second decision tree in the used random forest model according to the disclosure;

FIG. 4 is a graph of model performance evaluation of the developed prediction model on the validation set and the test set according to the disclosure; and

FIG. 5 is a graph of the SHAP model output of the developed prediction model according to the disclosure.

### DETAILED DESCRIPTION

To further illustrate, embodiments detailing a method for evaluating bioavailability of organic nitrogen in sewage through machine learning are described below. It should be noted that the following embodiments are intended to describe and not to limit the disclosure.

### Example 1

Sewage samples from a sewage plant were selected to evaluate the biodegradability of soluble organic nitrogen, where the average value of the COD concentration of the sewage is 150.1 mg/L, the average value of the total nitrogen concentration is 16.2 mg/L, the average value of the organic nitrogen concentration is 3.2 mg/L, and the average value of the total phosphorus concentration is 1.1 mg/L. The specific evaluation steps are described below.

(1) 100 pieces of the molecular composition information of organic nitrogen in sewage measured by a Fourier transform ion cyclotron resonance mass spectrometer and the bioavailability data measured by the algae biological culture are collected.

(2) The organic nitrogen molecular parameter of each sewage sample is calculated as a feature value. The specific calculation process is described below.

The molecular parameters of all organic molecules are as follows: the average value of the mass-to-charge ratio (m/z) to obtain a feature vector x₁ = (x₁₁; x₁₂; x₁₃; ...; x₁ₙ); the average value of the number of carbon atoms (C) to obtain a feature vector x₂ = (x₂₁; x₂₂; x₂₃; ...; x₂ₙ); the average value of the number of hydrogen atoms (H) to obtain a feature vector x₃ = (x₃₁; x₃₂; x₃₃; ...; x₃ₙ); the average value of the number of oxygen atoms (O) to obtain a feature vector x₄ = (x₄₁; x₄₂; x₄₃; ...; x₄ₙ); the average value of the number of nitrogen atoms (N) to obtain a feature vector x₅ = (x₅₁; x₃₂; x₅₃; ...; x₅ₙ); the average value of the ratio of the number of oxygen atoms to the number of carbon atoms (O/C) to obtain a feature vector x₆= (x₆₁; x₆₂; x₆₃; ...; x₆ₙ); the average value of the ratio of the number of hydrogen atoms to the number of carbon atoms (H/C) to obtain a feature vector x₇= (x₇₁; x₇₂; x₇₃; ...; x₇ₙ); the average value of the number of double bond equivalents (DBE)

$$\left(\text{i} \text{.e} \text{., DBE=} \frac{2 \text{C+N+P-H+2}}{2}\right)$$

) to obtain a feature vector x₈ = (x₈₁; x₈₂; x₈₃; ...; x₈ₙ); the average value of the ratio of the number of double bond equivalents to the number of hydrogen atoms (DBE/H) to obtain a feature vector x₉ = (x₉₁; x₉₂; x₉₃; ...; x₉ₙ); the average value of the ratio of the number of double bond equivalents to the number of oxygen atoms (DBE/O) to obtain a feature vector x₁₀ = (x₁₀₁; x₁₀₂; x₁₀₃; ...; x₁₀ₙ); the average value of the ratio of the difference between the number of double bond equivalents and the number of oxygen atoms to the number of carbon atoms ((DBE-O)/C) (i.e.,

$$\frac{\text{DBE} - \text{O}}{\text{C}} = \frac{\left(2 \text{C+N+P} - \text{H+2}\right) / 2 - \text{O}}{\text{C}}$$

to obtain a feature vector x₁₁ = (x₁₁₁; x₁₁₂; x₁₁₃; ...; x₁₁ₙ); the average value of the nominal oxidation state of carbon (NOSC)

$$\left(\text{i} \text{.e} \text{., NOSC} = 4 - \frac{4 \text{C+H} - \text{20} - \text{3N} - \text{2S+5P}}{\text{C}}\right)$$

^{4C+H-20-3N-2S+SP}) to obtain a feature vector x₁₂ = (x₁₂₁; x₁₂₂; x₁₂₃; ...; x₁₂ₙ); the sum of strength weighted average values of m/z (i.e., m/z_{wa} = ∑(m/zᵢ × RIᵢ)) to a feature vector x₁₃ = (x₁₃₁; x₁₃₂; x₁₃₃; ...; x₁₃ₙ); the sum of strength weighted average values of C (i.e., C_{wa} = Σ(Cᵢ × RIᵢ)) to obtain a feature vector x₁₄= (x₁₄₁; x₁₄₂; x₁₄₃; ...; x₁₄ₙ); the sum of strength weighted average values of H (i.e., H_{wa} = Σ(Hᵢ × RIᵢ)) to obtain a feature vector x₁₅ = (x₁₅₁; x₁₅₂; x₁₅₃; ...; x₁₅ₙ); the sum of strength weighted average values of O (i.e., O_{wa} = Σ(Oᵢ × RIᵢ)) to obtain a feature vector x₁₆= (x₁₆₁; x₁₆₂; x₁₆₃; ...; x₁₆ₙ); the sum of strength weighted average values of N (i.e., N_{wa} = Σ(Nᵢ × RIᵢ)) to obtain a feature vector x₁₇= (x₁₇₁; x₁₇₂; x₁₇₃; ...; x₁₇ₙ); the sum of strength weighted average values of O/C (i.e., O/C_{wa} = Σ(O/Cᵢ × RIᵢ)) to obtain a feature vector x₁₈= (x₁₈₁; x₁₈₂; x₁₈₃; ...; x₁₈ₙ); the sum of strength weighted average values of H/C (i.e., H/C_{wa} = Σ(H/Cᵢ × RIᵢ)) to obtain a feature vector x₁₉ = (x₁₉₁; x₁₉₂; x₁₉₃; ...; x₁₉ₙ); the sum of strength weighted average values of DBE (i.e., DBE_{wa} = Σ(DBEᵢ × RIᵢ)) to obtain a feature vector x₂₀ = (x₂₀₁; x₂₀₂; x₂₀₃; ...; x₂₀ₙ); the sum of strength weighted average values of DBE/H (i.e., DBE/H_{wa} = Σ(DBE/Hᵢ × RIᵢ)) to obtain a feature vector x₂₁ = (x₂₁₁; x₂₂₂; x₂₃₃; ... ;x₂₄ₙ); the sum of strength weighted average values of DBE/O (i.e., DBE/H_{wa} = Σ(DBE/Hᵢ × RIᵢ)) to obtain a feature vector x₂₂ = (x₂₂₁; x₂₂₂; x₂₂₃; ... ;x₂₂ₙ); the sum of strength weighted average values of (DBE-O)/C (i.e., (DBE - O)/C_{wa} = Σ((DBE - O)/Cᵢ × RIᵢ)) to obtain a feature vector x₂₃ = (x₂₃₁; x₂₃₂; x₂₃₃; ...; x₂₃ₙ); and, the sum of strength weighted average values of NOSC (i.e., NOSC_{wa} = Σ(NOSCᵢ × RIᵢ)) to obtain a feature vector x₂₄ = (x₂₄₁; x₂₄₂; x₂₄₃; ... ; x₂₄ₙ).

All organic nitrogen molecules in each sample are classified into 7 molecule categories. By taking the calculation process of lipids as an example, the molecular parameters of all molecules in this molecule category are calculated as follows: the average value of m/z to obtain a feature vector x₂₅ = (x₂₅₁; x₂₅₂; x₂₅₃; ...; x₂₅ₙ); the average value of DBE to obtain a feature vector x₂₆= (x₂₆₁; x₂₆₂; x₂₆₃; ...; x₂₆ₙ); the average value of NOSC to obtain a feature vector x₂₇= (x₂₇₁; x₂₇₂; x₂₇₃; ...; x₂₇ₙ); the sum of strength weighted average values of m/z to obtain a feature vector x₂₈ = (x₂₈₁; x₂₈₂; x₂₈₃; ...; x₂₈ₙ); the sum of strength weighted average values of DBE to obtain a feature vector x₂₉= (x₂₉₁; x₂₉₂; x₂₉₃; ...; x₂₉ₙ); the sum of strength weighted average values of NOSC to obtain a feature vector x₃₀ = (x₃₀₁; x₃₀₂; x₃₀₃; ...; x₃₀ₙ); and, the ratio Num₁ of the number of molecules of this category in the number of all molecules in this sample to obtain a feature vector x₃₁= (x₃₁₁; x₃₁₂; x₃₁₃; ...; x₃₁ₙ). The calculation process of other six molecule categories is the same as above and would not be repeated here.

(3) 73 features values obtained for each sewage sample are merged, and there are totally 100 sewage samples, so 100 original sample sets (x₁, x₂, x₃, ..., x₁₀₀) are obtained. Data standardization is performed on the calculated feature values by the following calculation formula:

$$z = \frac{\left(x - u\right)}{s} ;$$

where z is the standardized feature value, x is the original feature value, u is the average value of the feature value, and s is the standard deviation of the feature value. The data of the bioavailability of organic nitrogen in the sewage samples is incorporated into the standardized original sample sets to obtain an original data set D = ((x₁, y₁), (x₂, y₂), (x₃,y₃), ..., (x₁₀₀, y₁₀₀)) = (((-0.137, 0.284, 2.077, ..., -0.692)^{T}, 48.4), ((-0.912, -0.217, 0.910, ..., -0.532)^{T}, 58.9), ((0.556, 0.240, -0.148, ..., -0.315)^{T}, 30), ..., ((0.407, 0.218, 0.028, ..., -0.393)^{T}, 30)).

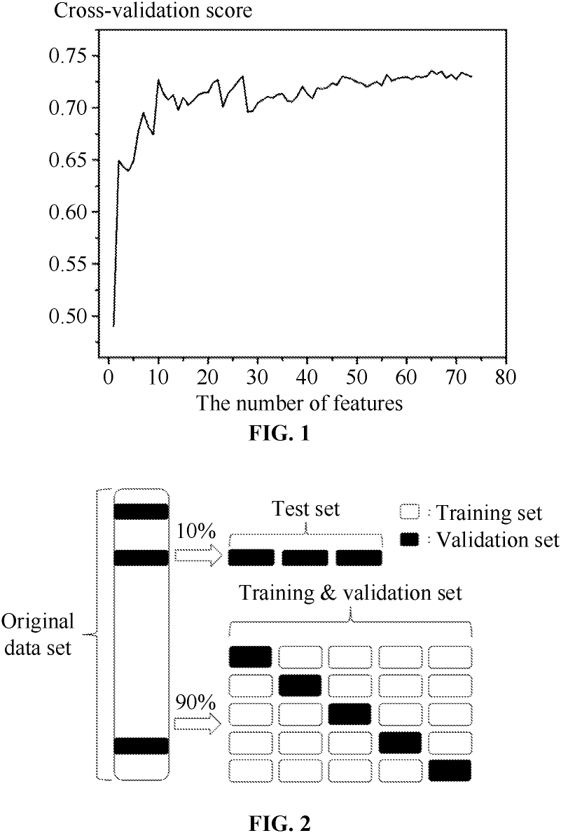

(4) The best number of features was searched, and features to be deleted were determined by feature ranking. In order to search the best number of features, a recursive feature elimination algorithm with cross-validation is used, NGBoost is selected as a learning estimator, and a determination coefficient R² is used as a cross-validation scoring basis. Every time one feature number is removed from the current feature set, the feature elimination process is recursively circulated in the updated feature set until the cross-validation score of the model decreased due to the feature elimination, and features to be deleted are determined by feature ranking. As shown in FIG. 1, the best number of features is determined as 65, and the features to be deleted are determined by feature ranking.

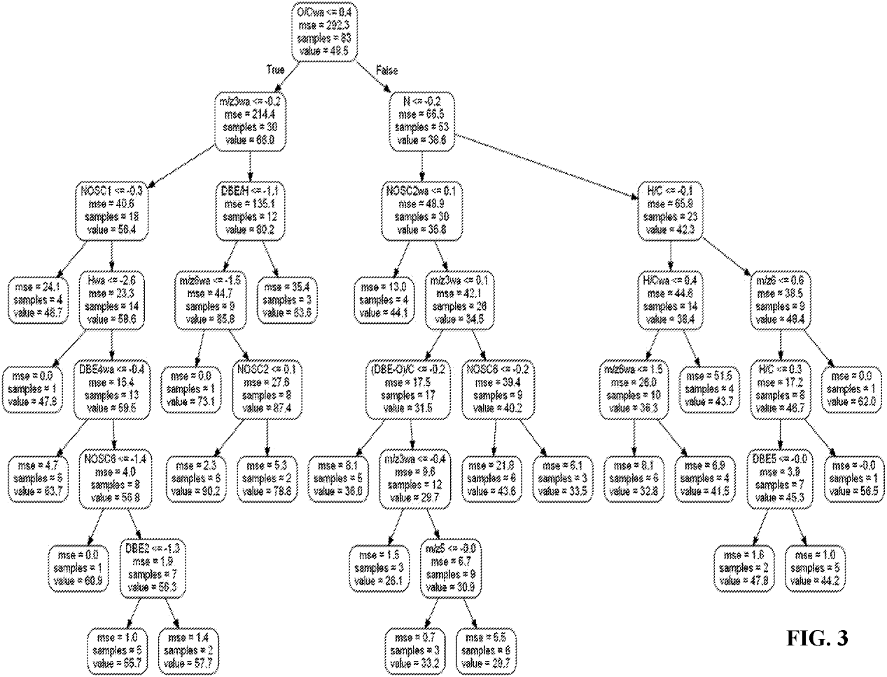

(5) The data set is randomly divided into a training set and a test set at a ratio of 9:1. As shown in FIG. 2, m samples are randomly selected from the given training set to construct a sample set, k attributes are randomly selected from an attribute set of each node of a base decision tree by using a decision tree as a base learner, and the best attribute is selected from the k attributes for division. Sampling is performed for T times to construct a sample set containing m training samples, and one decision tree is trained based on each sample set, as shown in FIG. 3. A random forest model is formed based on T decision trees, and the final predicted value of the random forest model can be expressed as:

$${f}^{⌣} \left(x\right) = \frac{1}{T} {\sum }_{i = 1}^{K} T \left(x\right) ;$$

where f̌(x) is the final predicted value of the random forest model, T is the number of decision trees, and T(x) is the output value of each decision tree. The training set employs a 5-fold cross-validation mode to adjust model parameters and train the model, and the adjusted model hyper-parameters are evaluated on the validation set. The parameters to be adjusted and the ranges thereof are as follows: the number of base decision trees is 100 to 10000, the maximum depth of the decision tree is 5 to 55, and the minimum impurity reduction threshold is 0.0 to 0.1. The parameters are randomly matched and combined to output the best parameter combination. Based on the randomly matched best parameter combination, a number of values are selected in the parameter proximity range, and all possible combinations of parameters are traversed to output the best parameter combination. The number of final best parameter combinations is equal to the number of base decision trees, i.e., 100; the maximum depth of the decision tree is 15; and, the minimum impurity reduction is 0.05. The model is trained by using the parameter combination on the training set through 5-fold cross-validation.

(6) The parameter combination with the best performance is selected to evaluate the performance of the random forest model on the test set. The determination coefficient R² and the root mean square error RMSE are selected as evaluation indexes, wherein the calculation formula of R² is as follows:

$${R}^{2} \left(y , {y}^{⌣}\right) = 1 - \frac{{\sum }_{i = 1}^{n} {\left({y}_{i} - {{y}^{⌣}}_{i}\right)}^{2}}{{\sum }_{i = 1}^{n} {\left({y}_{i} - \overline{y}\right)}^{2}} ;$$

where yᵢ is the real value, y̌ᵢ is the predicted value, and

$$\overline{y} = \frac{1}{n} {\sum }_{i = 1}^{n} {y}_{i} .$$

the calculation formula of the RMSE is:

$$R M S E = \sqrt{\frac{1}{n} {\sum }_{i = 1}^{n} {\left({{y}^{⌣}}_{i} - {y}_{i}\right)}^{2}} .$$

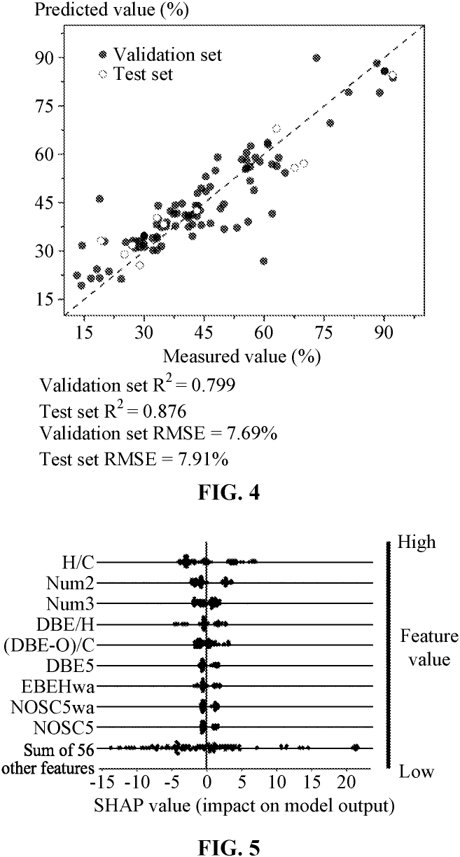

(7) Finally, as shown in FIG. 4, the trained machine learning prediction model has an R² of 0.779 and an RMSE of 7.69% on the validation set, and has an R² of 0.879 and an RMSE of 7.91% on the test set. In addition, there is no significant difference between the predicted value and the experimental value obtained by algae biological culture, as shown in the following table:

| Difference source | SS | df | MS | F | P-value | F crit |
|---|---|---|---|---|---|---|
| Column | 0.409771 | 1 | 0.409771 | 0.013102 | 0.910931 | 4.844336 |
| Error | 344.0186 | 11 | 31.27442 | | | |
| Total | 344.42837 | 12 | | | | |

The established prediction model is interpreted by a SHAP model. As shown in FIG. 5, the results have shown that the importance of features and the rule of influence on the bioavailability of organic nitrogen are basically consistent with the conclusions in the document, indicating that the prediction model has good prediction performance and high credibility.

(8) The molecular composition of organic nitrogen in printing and dyeing wastewater samples is measured by the Fourier transform ion cyclotron resonance mass spectrometer.

(9) According to the requirements in (4), the desired feature values are extracted to obtain a feature vector X = (x₁; x₂; x_{3;} ...; x₆₅); and, standardization is performed according to the mean values and variances of the respective feature values in the original data set to obtain a standardized feature vector X = (0.056; -0.138; -0.127; ...; -0.323)^{T}.

(10) The feature vector X is input into the trained machine learning prediction model, and the prediction model is run to obtain an output value of 44.6%. The bioavailability of organic nitrogen in the water samples is 43.2% obtained by algae biological culture. In accordance with the disclosure, there is no significant difference between the predicted value of the bioavailability of organic nitrogen in sewage obtained by using the machine learning model and the numerical value of the DON bioavailability measured by algae biological culture, and the prediction accuracy is 96.8%.

### Example 2

Sewage samples from a sewage plant are selected to evaluate the biodegradability of soluble organic nitrogen, where the average value of the COD concentration of the samples is 35.4 mg/L, the average value of the total nitrogen concentration is 12.8 mg/L, the average value of the organic nitrogen concentration is 0.9 mg/L, and the average value of the total phosphorus concentration is 0.09 mg/L. The specific evaluation steps were described below.

(1) The model establishment process is the same as that in Embodiment 1.

(2) The molecular composition of soluble organic nitrogen in pharmaceutical sewage samples is measured by the Fourier transform ion cyclotron resonance mass spectrometer.

(3) The desired feature values are extracted to obtain a feature vector X = (x₁; x₂; x₃; ...; x₆₅); and, standardization is performed according to the mean values and variances of the respective feature values in the original data set to obtain a standardized feature vector X= (-0.032; -0.284; 2.60; ...; -0.571)^{T}.

(4) The feature vector X is input into the trained machine learning prediction model, and the prediction model is run to obtain an output value of 84.6%. The bioavailability of organic nitrogen in the water samples is 92.1% obtained by algae biological culture. In accordance with the disclosure, there is no significant difference between the predicted value of the bioavailability of organic nitrogen in sewage obtained by using the machine learning model and the numerical value of the DON bioavailability measured by algae biological culture, and the prediction accuracy is 91.9%.

It will be obvious to those skilled in the art that changes and modifications may be made, and therefore, the aim in the appended claims is to cover all such changes and modifications.

The invention claimed is:

1. A method, comprising:
(1) collecting molecular composition information and bioavailability data of organic nitrogen in a sewage sample;
(2) establishing a model for predicting bioavailability of organic nitrogen in sewage through machine learning;
(3) measuring molecular composition information of organic nitrogen in sewage from a target sewage plant; and
(4) predicting, according to the model established in (2), the bioavailability of the organic nitrogen in the sewage from the target sewage plant.

2. The method of claim 1, wherein in (1), the molecular composition information of organic nitrogen in the sewage sample comes from data measured by a Fourier transform ion cyclotron resonance mass spectrometer, and the bioavailability of organic nitrogen in sewage comes from data measured by algae biological culture.

3. The method of claim 1, wherein in (2), the model for predicting bioavailability of organic nitrogen in sewage is established by a random forest model in machine learning, which comprises:
(a) calculating an organic nitrogen molecular parameter, and performing data standardization by using the organic nitrogen molecular parameter as a feature value;
(b) searching a best number of features, and determining features to be deleted by feature ranking;
(c) dividing a data set to obtain a training set, a validation set, and a test set, training the model by using the training set, and optimizing model parameters by using the validation set; and
(d) selecting a best optimal model parameter to train the model to obtain a prediction model, and evaluating the performance of the prediction model by using the test set.

4. The method of claim 3, wherein
in (a), the organic nitrogen molecular parameter as the feature value comprises: molecular parameters of all organic nitrogen molecules, and organic nitrogen molecular parameters of seven molecule categories;
the molecular parameters of all organic nitrogen molecules comprise: a mass-to-charge ratio m/z of all organic nitrogen molecules, a number C of carbon atoms of all organic nitrogen molecules, a number H of hydrogen atoms of all organic nitrogen molecules, a number O of oxygen atoms of all organic nitrogen molecules, a number N of nitrogen atoms of all organic nitrogen molecules, a ratio O/C of the number of oxygen atoms to the number of carbon atoms, a ratio H/C of the number of hydrogen atoms to the number of carbon atoms, a number DBE of double bond equivalents, a ratio DBE/H of the number of double bond equivalents to the number of hydrogen atoms, a ratio DBE/O of the number of double bond equivalents to the number of oxygen atoms, a ratio (DBE-O)/C of a difference between the number of double bond equivalents and the number of oxygen atoms to the number of carbon atoms, an average value of a nominal oxidation state of carbon (NOSC) of all organic nitrogen molecules, and strength weighted average values of molecular parameters, which are equal to a sum of products of respectively multiplying corresponding relative peak strength of molecules by m/z, C, H, O, N, O/C, H/C, DBE, DBE/H, DBE/O, (DBE-O)/C and NOSC;
the seven molecule categories are: lipids, proteins/amino sugars, carbohydrates, unsaturated hydrocarbons, lignin, tannins and condensed aromatics; screening conditions for lipids are as follows: O/C < 0.2 and 1.7 < H/C < 2.2; screening conditions for proteins/amino sugars are as follows: 0.2 < O/C < 0.6, 1.5 < H/C < 2.2 and N/C ≥ 0.05; screening conditions for carbohydrates are as follows: 0.6 < O/C < 1.0 and 1.5 < H/C < 2.2; screening conditions for unsaturated hydrocarbons are as follows: O/C<0.1, 0.7<H/C<1.5; screening conditions for lignin are as follows: 0.1 < O/C < 0.6, 0.6 < H/C < 1.7, and a modified aromaticity index AImod < 0.67; screening conditions for tannins are as follows: 0.6 < O/C < 1.0, 0.5 < H/C < 1.5 and a modified aromaticity index AImod < 0.67; and, screening conditions for condensed aromatics are as follows: O/C < 1.0, 0.3 < H/C < 0.7 and a modified aromaticity index AImod ≥ 0.67; and
the organic nitrogen molecular parameters of seven molecule categories comprise: a mass-to-charge ratio m/zi of seven molecule categories, a number DBEi of double bond equivalents of seven molecule categories, an average value of a nominal oxidation state of carbon NOSCi of organic nitrogen molecules of the seven molecule categories, a proportion Numi of the number of molecules in each category, and strength weighted average values of molecular parameters, which are equal to a sum of products of respectively multiplying corresponding relative peak strength of molecules by m/zi, DBEi and NOSCi, which i represents the molecule categories.

5. The method of claim 3, wherein the data standardization is performed on the feature value by the following calculation formula:
$$z = \frac{\left(x - u\right)}{s} ;$$
where z is a standardized feature value, x is an original feature value, u is an average value of the feature value, and s is a standard deviation of the feature value.

6. The method of claim 3, wherein in (b), to search the best number of features, a recursive feature elimination algorithm with cross-validation is used, NGBoost is selected as a learning estimator, and a determination coefficient R² is used as a cross-validation scoring basis; and, every time one feature number is removed from the current feature set, the feature elimination process is recursively circulated in the updated feature set until the cross-validation score of the model decreases due to the feature elimination, and features to be deleted are determined by feature ranking.

7. The method of claim 3, wherein
in (c), the data set is randomly divided into a training set and a test set at a ratio of 9:1, m samples are randomly selected from the given training set to construct a sample set, k attributes are randomly selected from an attribute set of each node of a base decision tree by using a decision tree as a base learner, and the best attribute is selected from the k attributes for division; sampling is performed for T times to construct a sample set containing m training samples, and one decision tree is trained based on each sample set; and, a random forest model is formed based on T decision trees, and a final predicted value of the random forest model is expressed as:
$${f}^{⌣} \left(x\right) = \frac{1}{T} {\sum }_{i = 1}^{T} T \left(x\right) ;$$
where f̌(x) is the final predicted value of the random forest model, T is a number of decision trees, and T(x) is the output value of each decision tree; the training set employs a 5-fold cross-validation mode to adjust model parameters and train the model, and the adjusted model hyper-parameters are evaluated on the validation set.

8. The method of claim 7, wherein the random forest parameters to be adjusted and the ranges thereof are as follows: a number of base decision trees is 100 to 10000, a maximum depth of the decision trees is 5 to 55, a minimum impurity reduction threshold is 0.0 to 0.1; the parameters are randomly matched and combined to output the best parameter combination; and, based on the randomly matched best parameter combination, a number of values are selected in the parameter proximity range, and all possible combinations of parameters are traversed to output the best parameter combination.

9. The method of claim 3, wherein
the best optimal model parameter is selected to train the model to obtain a prediction model, and the performance of the prediction model is evaluated by using the test set; and, the determination coefficient R² and a root mean square error RMSE are selected as evaluation indexes, where the calculation formula of R² is:
$${R}^{2} \left(y , {y}^{⌣}\right) = 1 - \frac{{\sum }_{i = 1}^{n} {\left({y}_{i} - {{y}^{⌣}}_{i}\right)}^{2}}{{\sum }_{i = 1}^{n} {\left({y}_{i} - \overline{y}\right)}^{2}} ;$$
the calculation formula of the RMSE is:
$$R M S E = \sqrt{\frac{1}{n} {\sum }_{i = 1}^{n} {\left({{y}^{⌣}}_{i} - {y}_{i}\right)}^{2}} ;$$
where yᵢ is a real value, y̌ᵢ is a predicted value,
$$\overline{y} = \frac{1}{n} {\sum }_{i = 1}^{n} {y}_{i} , \text{and} n \text{is}$$
a total number of sewage samples.

10. The method of claim 1, wherein using the model to predict the bioavailability of the organic nitrogen in the sewage comprises:
(a) measuring the molecular composition information of organic nitrogen in sewage by a Fourier transform ion cyclotron resonance mass spectrometer;
(b) calculating a desired feature value, and performing data standardization on the feature value; and
(c) inputting the data in (b) into the prediction model, and running the prediction model to obtain an output vale, so that the prediction of the bioavailability of organic nitrogen in sewage can be completed.
